(19) United States
(12) Patent Application Publication
(10) Pub. No.: US 2026/0108303 A1
(43) Pub. Date: Apr. 23, 2026

(54) POWER AND DATA INTERFACE FOR INTRAVASCULAR LITHOTRIPSY CATHETER SYSTEM

(71) Applicant: BOLT MEDICAL, INC., Carlsbad, CA (US)

(72) Inventor: Lakshmi Narayan Mishra, Carlsbad, CA (US)

(73) Assignee: BOLT MEDICAL, INC., Carlsbad, CA (US)

(21) Appl. No.: 19/365,957

(22) Filed: Oct. 22, 2025

Related U.S. Application Data
(60) Provisional application No. 63/710,209, filed on Oct. 22, 2024.

(51) Int. Cl. A61B 18/26; A61B 17/00; A61B 18/00

(52) U.S. Cl. CPC A61B 18/26; A61B 2017/00199; A61B 2017/00221; A61B 2017/00411; A61B 2017/00734; A61B 2018/0022; A61B 2018/00369; A61B 2018/266

(57) ABSTRACT

A catheter system usable for treating a treatment site within or adjacent to a vessel wall of a blood vessel or a heart valve within a body of a patient, the catheter system can include an energy source that generates energy, a catheter that includes an energy guide that receives the energy from the energy source, and a system console that is wirelessly couplable to the catheter so that one of data and power can be exchanged between the system console and the catheter.

### CROSS-REFERENCE TO RELATED APPLICATIONS

This application claims the benefit of priority under 35 U.S. C. § 119 to U.S. Provisional Application No. 63/710,209, filed Oct. 22, 2024, the entire disclosure of which is hereby incorporated by reference herein for all purposes.

### BACKGROUND

Vascular lesions (also referred to herein as a “treatment site”) within and/or adjacent to vessels in the body can be associated with an increased risk for major adverse events, such as myocardial infarction, embolism, deep vein thrombosis, stroke, and the like. Severe vascular lesions, such as severely calcified vascular lesions, can be difficult to treat and achieve patency for a physician in a clinical setting.

Vascular lesions may be treated using interventions such as drug therapy, balloon angioplasty, atherectomy, stent placement, vascular graft bypass, to name a few. Such interventions may not always be ideal or may require subsequent treatment to address the lesion.

Intravascular lithotripsy is one method that has been recently used with some success for breaking up vascular lesions within vessels in the body. Intravascular lithotripsy utilizes a combination of pressure waves and bubble dynamics that are generated intravascularly in a fluid-filled balloon catheter. In particular, in certain implementations of an intravascular lithotripsy treatment, a high energy source may used to provide energy to an energy guide and/or an emitter in order to generate plasma and, ultimately, pressure waves as well as a rapid bubble expansion within a fluid-filled balloon to crack calcification at a treatment site within the vasculature that includes one or more vascular lesions. The associated rapid bubble formation from the plasma initiation and resulting localized fluid velocity within the balloon transfers mechanical energy through the incompressible fluid to impart a fracture force on the intravascular calcium, which is opposed to the balloon wall. The rapid change in fluid momentum upon hitting the balloon wall is known as hydraulic shock, or water hammer.

### SUMMARY

The present disclosure is directed toward a catheter system usable for treating a treatment site within or adjacent to a vessel wall of a blood vessel or a heart valve within a body of a patient. In an example, the catheter system may include an energy source, a catheter, and a system console. The energy source may generate energy. The catheter may include an energy guide that receives the energy from the energy source. The system console may be wirelessly couplable to the catheter so that one of data and power can be exchanged between the system console and the catheter.

Alternatively or additionally to any of the examples above, only data can be exchanged between the system console and the catheter.

Alternatively or additionally to any of the examples above, only power can be exchanged between the system console and the catheter.

Alternatively or additionally to any of the examples above, both data and power can be exchanged between the system console and the catheter.

Alternatively or additionally to any of the examples above, the power may be transferred via one of a tuned coil and an antenna.

Alternatively or additionally to any of the examples above, the power may be transferred via one of Qi induction, WPC, and NFC power transfer.

Alternatively or additionally to any of the examples above, the data may be transferred via one of NFC, Bluetooth, BLE, Wi-Fi, Zigbee and ANT.

Alternatively or additionally to any of the examples above, two of NFC, Bluetooth, BLE, Wi-Fi, Zigbee and ANT may be used for increased data security.

Alternatively or additionally to any of the examples above, the system console may include (i) a transmitter, and (ii) and a receiver that receives one of data and power from the transmitter.

Alternatively or additionally to any of the examples above, the catheter may interface with the receiver so that the catheter can receive the one of data and power from the transmitter.

Alternatively or additionally to any of the examples above, the catheter may interface with the receiver via a console interface.

Alternatively or additionally to any of the examples above, the console interface may include (i) pads positioned on the catheter, and (ii) pogo pins positioned on the receiver, the pogo pins selectively engaging the pads.

Alternatively or additionally to any of the examples above, the catheter may be electrically isolated from the system console.

Alternatively or additionally to any of the examples above, the catheter may include a catheter battery that stores power and provides power to the catheter.

In an example, a catheter system may be usable for treating a treatment site within or adjacent to a vessel wall of a blood vessel or a heart valve within a body of a patient. The catheter system may include a catheter and a system console. The catheter may include a catheter battery that stores power and provides power to the catheter. The system console may be wirelessly couplable to the catheter so that one of data and power can be exchanged between the system console and the catheter. The system console may be electrically isolated from the catheter.

Alternatively or additionally to any of the examples above, only data can be exchanged between the system console and the catheter.

Alternatively or additionally to any of the examples above, only power can be exchanged between the system console and the catheter.

Alternatively or additionally to any of the examples above, both data and power can be exchanged between the system console and the catheter.

Alternatively or additionally to any of the examples above, the power may be transferred via one of a tuned coil and an antenna.

Alternatively or additionally to any of the examples above, the power may be transferred via one of Qi induction, WPC, and NFC power transfer.

Alternatively or additionally to any of the examples above, the data may be transferred via one of NFC, Bluetooth, BLE, Wi-Fi, Zigbee and ANT.

Alternatively or additionally to any of the examples above, two of NFC, Bluetooth, BLE, Wi-Fi, Zigbee and ANT may be used for increased data security.

Alternatively or additionally to any of the examples above, the system console may include (i) a transmitter, and (ii) and a receiver that receives one of data and power from the transmitter.

Alternatively or additionally to any of the examples above, the catheter may interface with the receiver so that the catheter can receive the one of data and power from the transmitter.

In an example, a catheter system may be usable for treating a treatment site within or adjacent to a vessel wall of a blood vessel or a heart valve within a body of a patient. The catheter system may include an energy source, a catheter, and a system console. The energy source may generate energy. The catheter may include an energy guide that receives the energy from the energy source. The catheter may include a catheter battery that stores power and provides power to the catheter. The system console may be wirelessly couplable to the catheter so that data and power can be exchanged between the system console and the catheter. The system console may be electrically isolated from the catheter.

In an example, a catheter system may include a shaft, a balloon coupled with the shaft, an energy guide extending through the shaft and into the balloon that receives energy from an energy source, an emitter coupled to the shaft and the energy guide, wherein the emitter is configured to transmit energy from the energy source into an interior of the balloon for treating the treatment site, and a catheter communicator module configured to be wirelessly connected to a system console via a wireless connection such that the catheter exchanges at least one of data and power with the system console via the wireless connection.

Alternatively or additionally to any of the examples above, the catheter communicator module may be configured to exchange only data with the system console via the wireless connection.

Alternatively or additionally to any of the examples above, the catheter communicator module may be configured to exchange only power with the system console via the wireless connection.

Alternatively or additionally to any of the examples above, the catheter communicator module may be configured to exchange data and power with the system console via the wireless connection.

Alternatively or additionally to any of the examples above, the catheter may further includes a catheter battery.

Alternatively or additionally to any of the examples above, the catheter battery may be configured to provide power to the catheter.

Alternatively or additionally to any of the examples above, the catheter battery may be rechargeable.

In an example, a system console for treating a treatment site within or adjacent to a vessel wall of a blood vessel or a heart valve within a body of a patient may include a power source configured to provide power, an energy source configured to receive energy from the power source, wherein the energy source is configured to transmit energy to an energy guide of a catheter for treating the treatment site, and a console communicator module configured to be wirelessly connected to a catheter communicator module of the catheter via a wireless connection such that the system console is configured to wirelessly exchange at least one of data and power with the catheter via the wireless connection.

Alternatively or additionally to any of the examples above, the console communicator module may be configured to exchange only data with the catheter via the wireless connection.

Alternatively or additionally to any of the examples above, the console communicator module is configured to exchange only power with the catheter via the wireless connection.

Alternatively or additionally to any of the examples above, the console communicator module may be configured to exchange data and power with the catheter via the wireless connection.

In an example, a catheter system for treating a treatment site within or adjacent to a vessel wall of a blood vessel or a heart valve within a body of a patient may include a system console including an energy source and a console communicator module, and a catheter that includes a shaft, a balloon coupled with the shaft, and an energy guide, wherein the energy guide receives energy from the energy source and transmits the energy to an interior of the balloon for treating the treatment site, and a catheter communicator module is wirelessly connected to the console communicator module of the system console via a wireless connection such that the system console exchanges at least one of data and power with the catheter via the wireless connection.

Alternatively or additionally to any of the examples above, the catheter communicator module may be included in the catheter.

Alternatively or additionally to any of the examples above, the console communicator module may be a tuned coil, and the catheter communicator module may be an antenna.

Alternatively or additionally to any of the examples above, the catheter communicator module may be included in the system console, and the catheter may be configured to interface with a console interface connected to the catheter communicator module in the system console.

Alternatively or additionally to any of the examples above, data may be exchanged via a wireless interface including at least one of near field communication (NFC), Bluetooth, Bluetooth Low Energy (BLE), Wi-Fi, Zigbee, and Adaptive Network Topology (ANT).

Alternatively or additionally to any of the examples above, the data may be exchanged via at least two wireless data interfaces.

Alternatively or additionally to any of the examples above, the power may be exchanged via a wireless power transfer technique.

Alternatively or additionally to any of the examples above, the catheter may include a catheter connector interface at a proximal end of the catheter, and the system console includes a console connector interface.

Alternatively or additionally to any of the examples above, the catheter communicator module may be connected to the catheter connector interface, and the console communicator module may be connected to the console connector interface.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The features of this disclosure will be best understood from the accompanying drawings, taken in conjunction with the accompanying description, in which similar reference characters refer to similar parts, and in which:

FIG. 1 is a simplified schematic cross-sectional view illustration of an embodiment of a catheter system in accordance with various embodiments, the catheter system including a graphical user interface having features of the present disclosure;

FIG. 2 is a simplified schematic view illustration of an embodiment of a portion of the catheter system including a catheter and a system console;

FIG. 3 is a simplified schematic view illustration of another embodiment of a portion of the catheter system including a catheter and a system console; and FIG. 4 is a simplified schematic view illustration of yet another embodiment of a portion of the catheter system including a catheter and a system console.

While embodiments of the present disclosure are susceptible to various modifications and alternative forms, specifics thereof have been shown by way of examples and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a catheter configured to advance to a vascular lesion, such as a calcified vascular lesion or a fibrous vascular lesion, at a treatment site located within or adjacent to a vessel wall of a blood vessel or a heart valve within a body of a patient. As used herein, the terms “treatment site,” “intravascular lesion,” and “vascular lesion” are used interchangeably unless otherwise noted. As such, the intravascular lesions and/or the vascular lesions are sometimes referred to herein as “lesions.”

Use of a catheter system can include electrical checks to ensure device safety. In previous approaches, the catheter is directly connected to the system console electrically for power and data communication, as well as optically in order to receive laser light from a laser light source in the system console. Due to this direct electrical connection for transferring power and/or data, various electrical checks have to be performed. Examples of such electrical checks can include pressure sensor functionality, integrity of profile stored on the catheter, and/or redundant checks of activation button state (e.g., pressed or unpressed), among other types of electrical checks.

Power and data interfaces for intravascular lithotripsy catheter system, according to the disclosure, can allow for the catheter itself to be no longer directly electrically connected to the system console for data and/or power transfer. While the direct optical connection is maintained, the data and/or power connection can be achieved wirelessly, allowing for the electrical checks previously required for the direct connection to be bypassed.

Those of ordinary skill in the art will realize that the following detailed description of the present disclosure is illustrative only and is not intended to be in any way limiting. Other embodiments of the present disclosure will readily suggest themselves to such skilled persons having the benefit of this disclosure. Reference will now be made in detail to implementations of the present disclosure, as illustrated in the accompanying drawings. The same or similar nomenclature and/or reference indicators will be used throughout the drawings, and the following detailed description to refer to the same or like parts.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is recognized that such a development effort might be complex and time-consuming, but would nevertheless be a routine undertaking of engineering for those of ordinary skill in the art having the benefit of this disclosure.

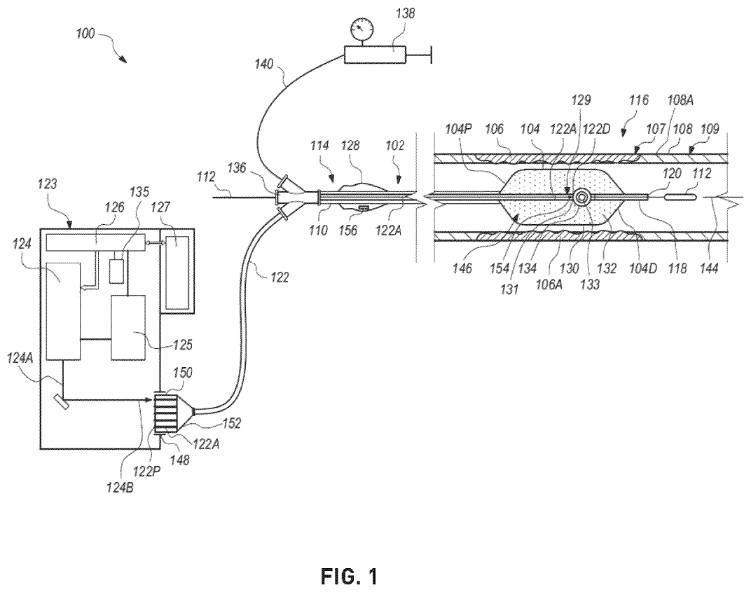

The catheter systems disclosed herein can include many different forms. Referring now to FIG. 1, a simplified schematic cross-sectional view illustration is shown of a catheter system 100 in accordance with various embodiments. The catheter system 100 is suitable for imparting pressure waves to induce fractures at one or more treatment sites within or adjacent to a vessel wall of a blood vessel or adjacent to a heart valve within a body of a patient. In the embodiment illustrated in FIG. 1, the catheter system 100 can include one or more of a catheter 102 including an energy guide bundle 122 including one or more energy guides 122A, a handle assembly 128, and an emitter assembly 129, a source manifold 136, a fluid pump 138, and a system console 123 including one or more of an energy source 124, a power source 125, a system controller 126, and a graphic user interface 127 (a “GUI”). In various embodiments, the emitter assembly 129 includes and/or incorporates at least one emitter 131 that is configured to direct and/or concentrate energy toward one or more vascular lesions 106A at a treatment site 106 within or adjacent to a vessel wall 108A of a blood vessel 108 or a heart valve within a body of a patient. Alternatively, the catheter system 100 can include more components or fewer components than those specifically illustrated and described in relation to FIG. 1.

The catheter 102 is configured to move to the treatment site 106 within or adjacent to the vessel wall 108A of the blood vessel 108 or a heart valve within the body of the patient. The treatment site 106 can include one or more vascular lesions 106A such as calcified vascular lesions, for example. Additionally, or in the alternative, the treatment site 106 can include vascular lesions 106A, such as fibrous vascular lesions. Still alternatively, in some implementations, the catheter 102 can be used at a treatment site 106 within or adjacent to a heart valve within the body of the patient.

The catheter 102 can include an inflatable balloon 104 (sometimes referred to herein simply as a “balloon”), a catheter shaft 110, and a guidewire 112. The balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter 102 and/or the catheter shaft 110 can also include a guidewire lumen 118, which is configured to move over the guidewire 112. As utilized herein, the guidewire lumen 118 defines a conduit through which the guidewire 112 extends. The catheter shaft 110 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106. In some embodiments, the balloon proximal end 104P can be coupled to the catheter shaft 110, and the balloon distal end 104D can be coupled to the guidewire lumen 118. The catheter 102 can include all of the components shown in FIG. 1 that are distal to the guide proximal end 122P, and the system console 123 can include all of the components shown in FIG. 1 that are proximal to the guide proximal end 122P.

The balloon 104 includes a balloon wall 130 that defines a balloon interior 146. The balloon 104 can be selectively inflated with a catheter fluid 132 to expand from a deflated state suitable for advancing the catheter 102 through a patient's vasculature, to an inflated state (as shown in FIG. 1) suitable for anchoring the catheter 102 in position relative to the treatment site 106. Stated in another manner, when the balloon 104 is in the inflated state, the balloon wall 130 of the balloon 104 is configured to be positioned substantially adjacent to the treatment site 106. It is appreciated that although FIG. 1 illustrates the balloon wall 130 of the balloon 104 being shown spaced apart from the treatment site 106 of the blood vessel 108 or a heart valve when in the inflated state, this is done for ease of illustration. It is recognized that the balloon wall 130 of the balloon 104 will typically be substantially directly adjacent to and/or abutting the treatment site 106 when the balloon 104 is in the inflated state.

As described, in various embodiments, the catheter system 100 and/or the emitter assembly 129 can include the at least one emitter 131 that is configured to transmit energy from the energy source 124 into the balloon interior 146 in order to generate plasma and/or pressure waves in the catheter fluid 132 within the balloon interior 146. Each of the emitters 131 includes a guide distal end 122D of one of the energy guides 122A, which is positioned within the balloon interior 146, and a corresponding plasma target 133 (also sometimes referred to as a “plasma generating structure” or a “plasma generator”) that is positioned near, but typically spaced apart from, the guide distal end 122D. As referred to herein, the plasma target 133 or “plasma generator” can include and/or incorporate any suitable type of structure that is located at or near the guide distal end 122D of the energy guide 122A. Energy from the energy source 124 is directed toward and received by the energy guide 122A, is guided through the energy guide 122A, and is then emitted from the guide distal end 122D of the energy guide 122A. The energy emitted from the guide distal end 122D is directed toward and impinges on and energizes the corresponding plasma target 133 for purposes of generating the plasma in the catheter fluid 132 within the balloon interior 146.

In many embodiments, the present disclosure utilizes a laser light source or other suitable light source as the energy source 124, and is configured to shine laser light energy onto the plasma target 133 to cause plasma generation via interaction with a plasma target material rather than optical breakdown of the catheter fluid 132. This moves the plasma creation away from the guide distal end 122D of the energy guide 122A (which can be an optical fiber in some embodiments). This can be accomplished by positioning the plasma target 133 away from the guide distal end 122D of the energy guide 122A to absorb the light energy and convert it into a plasma at some distance away from the guide distal end 122D of the energy guide 122A.

The balloon 104 suitable for use in the catheter system 100 includes those that can be passed through the vasculature of a patient 109 when in the deflated state. In some embodiments, the balloons 104 are made from silicone. In other embodiments, the balloon 104 can be made from materials such as polydimethylsiloxane (PDMS), polyurethane, polymers such as PEBAX™ material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In some embodiments, the balloon 104 can have a diameter (in the inflated state) ranging from at least two mm up to five mm.

In some embodiments, the balloon 104 can have a length ranging from at least three mm to 300 mm. More particularly, in some embodiments, the balloon 104 can have a length ranging from at least eight mm to 200 mm. It is appreciated that a balloon 104 having a relatively longer length can be positioned adjacent to larger treatment sites 106, and, thus, may be usable for imparting pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloon 104 can include a drug-eluting coating or a drug-eluting stent structure. The drug-eluting coating or drug-eluting stent can include one or more therapeutic agents including anti-inflammatory agents, anti-neoplastic agents, anti-angiogenic agents, and the like.

The catheter fluid 132 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the pressure waves are appropriately manipulated. In certain embodiments, the catheter fluids 132 suitable for use are biocompatible. A volume of catheter fluid 132 can be tailored by the chosen energy source 124 and the type of catheter fluid 132 used.

In certain embodiments, the catheter fluid 132 can include a wetting agent or surface-active agent (surfactant). These compounds can lower the tension between solid and liquid matter. These compounds can act as emulsifiers, dispersants, detergents, and water infiltration agents. Wetting agents or surfactants reduce surface tension of the liquid and allow it to fully wet and come into contact with optical components (such as the energy guide(s) 122A) and mechanical components (such as other portions of the emitter assembly 129). This reduces or eliminates the accumulation of bubbles and pockets or inclusions of gas within the emitter assembly 129. Nonexclusive examples of chemicals that can be used as wetting agents include, but are not limited to, Benzalkonium Chloride, Benzethonium Chloride, Cetylpyridinium Chloride, Poloxamer 188, Poloxamer 407, Polysorbate 20, Polysorbate 40, and the like. Non-exclusive examples of surfactants can include, but are not limited to, ionic and non-ionic detergents, and Sodium stearate. Another suitable surfactant is 4-(5-dodecyl) benzenesulfonate. Other examples can include docusate (dioctyl sodium sulfosuccinate), alkyl ether phosphates, and perfluorooctanesulfonate (PFOS), to name a few.

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium: yttrium-aluminum-garnet (Nd:YAG−emission maximum=1064 nm) lasers, holmium:YAG (Ho:YAG−emission maximum=2.1 μm) lasers, or erbium: YAG (Er:YAG−emission maximum=2.94 μm) lasers. In some embodiments, the absorptive agents can be water-soluble. In other embodiments, the absorptive agents are not water-soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the one or more energy guides 122A of the energy guide bundle 122 that are in optical communication with the energy source 124. The energy guide(s) 122A can be disposed along the catheter shaft 110 and within the balloon 104. In some embodiments, each energy guide 122A can be an optical fiber, and the energy source 124 can be a laser. The energy source 124 can be in optical communication with the energy guides 122A at the proximal portion 114 of the catheter system 100.

In some embodiments, the catheter shaft 110 can be coupled to multiple energy guides 122A, such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; or six energy guides 122A can be spaced apart from one another by approximately 60 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

In certain embodiments, the guidewire lumen 118 can have a grooved outer surface, with the grooves extending in a generally longitudinal direction along the guidewire lumen 118. In such embodiments, each of the energy guides 122A and/or the emitter(s) 131 of the emitter assembly 129 can be positioned, received, and retained within an individual groove formed along and/or into the outer surface of the guidewire lumen 118. Alternatively, the guidewire lumen 118 can be formed without a grooved outer surface, and the position of the energy guides 122A and/or the emitter(s) 131 of the emitter assembly 129 relative to the guidewire lumen 118 can be maintained in another suitable manner.

The catheter system 100 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100 and/or the energy guide bundle 122 can include from one energy guide 122A to greater than 30 energy guides 122A. Alternatively, in other embodiments, the catheter system 100 and/or the energy guide bundle 122 can include greater than 30 energy guides 122A.

The energy guides 122A can have any suitable design for the purposes of generating plasma and/or pressure waves in the catheter fluid 132 within the balloon interior 146. Thus, the general description of the energy guides 122A as light guides is not intended to be limiting in any manner, except for as set forth in the claims appended hereto. More particularly, although the catheter systems 100 are often described with the energy source 124 as a light source, and the one or more energy guides 122A as light guides, the catheter system 100 can alternatively include any suitable energy source 124 and energy guides 122A for purposes of generating the desired plasma in the catheter fluid 132 within the balloon interior 146. For example, in one non-exclusive alternative embodiment, the energy source 124 can be configured to provide high-voltage electrical pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the pressure waves in the catheter fluid 132 that are utilized to provide the fracture force onto the vascular lesions 106A at the treatment site 106. Still, alternatively, the energy source 124 and/or the energy guides 122A can have another suitable design and/or configuration.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P to the guide distal end 122D having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the length of the balloon 104 and/or relative to the length of the guidewire lumen 118 to more effectively and precisely impart pressure waves for purposes of disrupting the vascular lesions 106A at the treatment site 106.

In certain embodiments, the energy guides 122A can include one or more photoacoustic transducers 154, where each photoacoustic transducer 154 can be in optical communication with the energy guide 122A within which it is disposed. In some embodiments, the photoacoustic transducers 154 can be in optical communication with the guide distal end 122D of the energy guide 122A. In such embodiments, the photoacoustic transducers 154 can have a shape that corresponds with and/or conforms to the guide distal end 122D of the energy guide 122A.

The photoacoustic transducer 154 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 154 disposed at the guide distal end 122D of the energy guide 122A can assume the same shape as the guide distal end 122D of the energy guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 154 disposed along one or more side surfaces of the length of the energy guide 122A.

In some embodiments, the energy guides 122A and/or the emitter assembly 129 can further include one or more diverting structures or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting structure can include any structure of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can be configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing energy away from the guide distal end 122D of the energy guide 122A can include, but are not to be limited to, those having a convex surface, a gradient-index (GRIN) lens, and a mirror focus lens. Upon contact with the diverting structure, the energy is diverted within the energy guide 122A to one or more of the plasma target 133 and the photoacoustic transducer 154 that is in optical communication with a side surface of the energy guide 122A. When utilized, the plasma target 133 receives energy emitted from the guide distal end 122D of the energy guide 122A to generate plasma in the catheter fluid 132 within the balloon interior 146, which, in turn, causes the creation of plasma bubbles and/or pressure waves that can be directed away from the side surface of the energy guide 122A and toward the balloon wall 130. Additionally, or in the alternative, when utilized, the photoacoustic transducer 154 converts light energy into an acoustic wave that extends away from the side surface of the energy guide 122A.

Additionally, or in the alternative, in certain embodiments, such diverting structures that can be incorporated into the energy guides 122A, can also be incorporated into the design of the emitter assembly 129 and/or the plasma target 133 for purposes of directing and/or concentrating acoustic and mechanical energy toward specific areas of the balloon wall 130 in contact with the vascular lesions 106A at the treatment site 106 to impart pressure onto and induce fractures in such vascular lesions 106A.

The source manifold 136 can be positioned at or near the proximal portion 114 of the catheter system 100. The source manifold 136 can include one or more proximal end openings that can receive the one or more energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132 as needed.

As noted above, in the embodiment illustrated in FIG. 1, the system console 123 includes one or more of the energy source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, and the GUI 127 can be provided within the catheter system 100 without the specific need for the system console 123.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (also sometimes referred to generally as a “ferrule”) that houses a portion, such as the guide proximal end 122P, of each of the energy guides 122A. The guide coupling housing 150 is configured to fit and be selectively retained within the console connection aperture 148 to provide the mechanical coupling between the energy guide bundle 122 and the system console 123.

The energy guide bundle 122 can also include a guide bundler 152 (or “shell”) that brings each of the individual energy guides 122A closer together so that the energy guides 122A and/or the energy guide bundle 122 can be in a more compact form as it extends with the catheter 102 into the blood vessel 108 or the heart valve during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122, such as through the use of a multiplexer (not shown), as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104, such as via the plasma target 133 that can be located at or near the guide distal end 122D of the energy guide 122A. In particular, in such embodiments, the energy emitted at the guide distal end 122D of the energy guide 122A is directed toward and energizes the plasma target 133 to form the plasma in the catheter fluid 132 within the balloon interior 146. The plasma formation can initiate a pressure wave and can initiate the rapid formation of one or more bubbles that can rapidly expand to a maximum size and then dissipate through a cavitation event that can also launch a pressure wave upon collapse. An illustrative plasma-induced bubble 134 is illustrated in FIG. 1. The rapid expansion of the plasma-induced bubbles 134 can generate one or more pressure waves within the catheter fluid 132 and thereby impart pressure waves upon the treatment site 106. The pressure waves can transfer mechanical energy through an incompressible catheter fluid 132 to the treatment site 106 to impart a fracture force on the vascular lesions 106A at the treatment site 106. Without wishing to be bound by any particular theory, it is believed that the rapid change in catheter fluid 132 momentum upon the balloon wall 130 of the balloon 104 that is in contact with or positioned near the vascular lesions 106A at the treatment site 106 is transferred to the vascular lesions 106A to induce fractures in the vascular lesions 106A.

In various non-exclusive alternative embodiments, the sub-millisecond pulses of energy from the energy source 124 can be delivered to the treatment site 106 at a frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a frequency that can be greater than 5000 Hz or less than one Hz, or any other suitable range of frequencies.

It is appreciated that although the energy source 124 is typically utilized to provide pulses of energy, the energy source 124 can still be described as providing a single source beam 124A, i.e., a single pulsed source beam.

The energy sources 124 suitable for use can include various types of light sources including lasers and lamps. Alternatively, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths, and energy levels that can be employed to achieve plasma in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Illustrative nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 kHz.

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium: yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 in the catheter fluid 132.

The catheter system 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In various non-exclusive alternative embodiments, the catheter systems 100 can generate pressure waves having maximum pressures in the range of at least approximately two MPa to 50 MPa, at least approximately two MPa to 30 MPa, or approximately at least 15 MPa to 25 MPa.

The pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to 30 MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

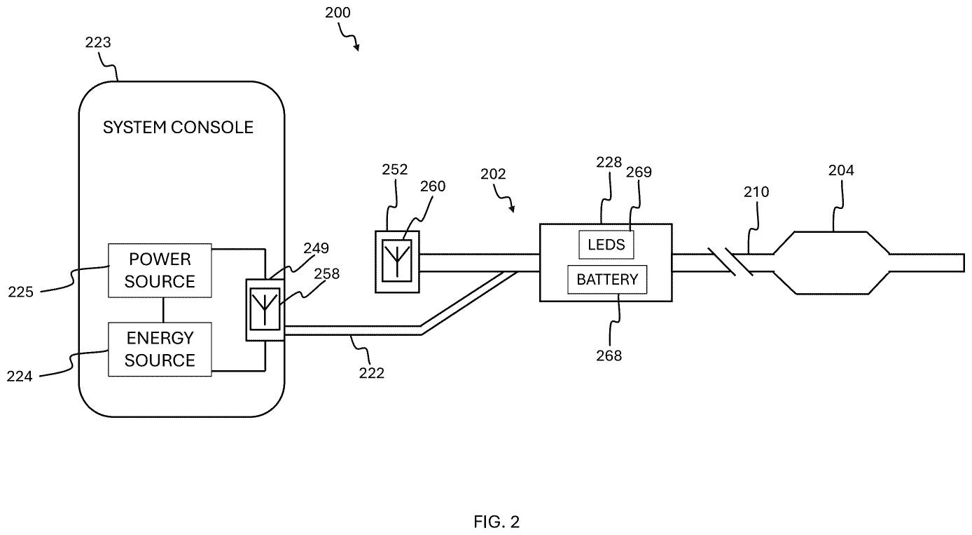

The power source 125 is electrically coupled to and is configured to provide necessary power to each of the energy source 124, the system controller 126, the GUI 127, and the catheter 102. The power source 125 can have any suitable design for such purposes. As is further described in connection with FIGS. 2-4, the power source 125 can wirelessly exchange power with the catheter 102.

The system controller 126 is electrically coupled to and receives power from the power source 125. The system controller 126 is coupled to and is configured to control operation of each of the energy source 124 and the GUI 127. The system controller 126 can include one or more processors or circuits for purposes of controlling the operation of at least the energy source 124 and the GUI 127. For example, the system controller 126 can control the energy source 124 for generating pulses of energy as desired and/or at any desired firing rate.

The system controller 126 can also be configured to control operation of other components of the catheter system 100, such as the positioning of the catheter 102 adjacent to the treatment site 106, the inflation of the balloon 104 with the catheter fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. The GUI 127 is communicatively coupled to the system controller 126. With such design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during, and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The GUI 127 can also provide the user or operator with control of other components of the catheter system 100. The specifics of the GUI 127 can vary depending upon the design requirements of the catheter system 100, or the specific needs, specifications, and/or desires of the user or operator.

As shown in FIG. 1, the handle assembly 128 can be positioned at or near the proximal portion 114 of the catheter system 100, and/or near the source manifold 136. In this embodiment, the handle assembly 128 is coupled to the balloon 104 and is positioned spaced apart from the balloon 104. Alternatively, the handle assembly 128 can be positioned at another suitable location.

The handle assembly 128 is handled and used by the user or operator to operate, position, and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, and the GUI 127. In some embodiments, the handle assembly 128 can integrate and/or include at least a portion of the system controller 126 within an interior of the handle assembly 128. For example, as shown, in certain such embodiments, the handle assembly 128 can include circuitry 156 that can form at least a portion of the system controller 126. In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, such as within the system console 123. It is understood that the handle assembly 128 can include fewer or additional components than those specifically illustrated and described herein.

In various embodiments, as noted above, the emitter assembly 129 includes and/or incorporates the at least one emitter 131 that is configured to transmit energy from the energy source 124 into the balloon interior 146 so that plasma and/or pressure waves are generated in the catheter fluid 132 within the balloon interior. Each emitter 131 includes the guide distal end 122D of one of the energy guides 122A and the corresponding plasma target 133 that is positioned near, but typically spaced apart from, the guide distal end 122D. Additionally, in many embodiments, each emitter 131 further includes an emitter housing that is configured to maintain the desired positioning between the guide distal end 122D of the energy guide 122A and the plasma target 133, and to direct and/or concentrate energy generated in the catheter fluid 132 within the balloon interior 146 so as to impart pressure onto and induce fractures in vascular lesions 106A at the treatment site 106.

During use of the catheter system 100, the energy guide 122A receives the energy from the energy source 124 and guides the energy from the guide proximal end 122P to the guide distal end 122D. The energy is then emitted from the guide distal end 122D of the energy guide 122A so that the energy is directed toward and impinges on and energizes the corresponding plasma target 133 for purposes of generating the plasma in the catheter fluid 132 within the balloon interior 146. The plasma generation then forms the pressure waves in the catheter fluid 132 that are directed toward the vascular lesions 106A at the treatment site 106 to provide the fracture force onto the vascular lesions 106A at the treatment site 106.

The plasma target 133 can be formed from any suitable material that is configured to generate the desired plasma in the catheter fluid 132 within the balloon interior 146 when the energy is directed from the guide distal end 122D of the energy guide 122A to impinge on the plasma target 133.

Various alternative embodiments of GUI 127 are illustrated and described in detail herein below within subsequent Figures.

As with all embodiments illustrated and described herein, various structures may be omitted from the figures for clarity and ease of understanding. Additionally, the figures may include certain structures that can be omitted without deviating from the intent and scope of the disclosure. It is further recognized that the structures included in the various figures shown and described herein are not necessarily drawn to scale for ease of viewing and/or understanding.

FIG. 2 is a simplified schematic view of an illustrative embodiment of a portion of the catheter system 200 including a catheter 202 and a system console 223. The catheter 202 and the system console 223 can be the same and/or similar to the catheter 102 and the system console 123, previously described in connection with FIG. 1, or different.

As illustrated in FIG. 2, the system console 223 can include an energy source 224 and a power source 225. As previously mentioned in FIG. 1, the energy source 224 can be, for example, a laser or other suitable source of energy. Additionally, the power source 225 can be electrically coupled to the energy source 224 and/or to a console connector 249. The console connector 249 can provide power to, for example, a console communicator module 258.

In some examples, the console communicator module 258 can be, for instance, a transceiver. The console communicator module 258 can be a device that can transmit and/or receive data and/or power. For example, the console communicator module 258 can wirelessly transmit and/or receive data and/or power with the catheter 202, as is further described herein.

In some examples, the console communicator module 258 can include a tuned coil. For instance, the console communicator module 258 can include a coil or inductor (or an interconnected arrangement of coils) that is adjusted to resonate at a specific frequency via the coil's inductance and capacitance allowing for receipt, transmission, and/or filtering of signals. The console communicator module 258 can, therefore, utilize resonant inductive coupling in order to wirelessly transmit and/or receive data and/or power with the catheter 202, as is further described herein. The console communication module 258 may have other suitable configurations for wirelessly transmitting and/or receiving data and/or power with the catheter 202.

As illustrated in FIG. 2, the catheter 202 can include a shaft 210, a balloon 204 coupled with the shaft 210, an energy guide (e.g., not illustrated in FIG. 2), and/or other suitable components. The catheter 202 can be optically connected to the system console 223 via the energy guide bundle 222. The energy guide can receive energy from the energy source 224 of the system console 223 (e.g., via the energy guide bundle 222) and transmit the energy to an interior of the balloon 204 for treating the treatment site. For example, the catheter 202 can include an emitter (e.g., not illustrated in FIG. 2) coupled to the shaft 210 and to the energy guide. The emitter can transmit the energy from the energy source 224 into the interior of the balloon for treating the treatment site, as previously described in connection with FIG. 1.

In the embodiment illustrated in FIG. 2, the catheter 202 can include a catheter connector 252. The catheter connector 252 can include a catheter communicator module 260. For example, in the embodiment illustrated in FIG. 2, the catheter 202 can include the catheter communicator module 260 in the catheter 202.

In some examples, the catheter communicator module 260 can be, for instance, a transceiver. The catheter communicator module 260 can be a device that can transmit and/or receive data and/or power. For example, the catheter communicator module 260 can wirelessly transmit and/or receive data and/or power with the system console 223, as is further described herein.

In some examples, the catheter communicator module 260 can include an antenna. For example, the antenna can transmit and/or receive electromagnetic waves in order to wirelessly transmit and/or receive data and/or power with the system console 223, as is further described herein.

As mentioned above, the catheter communicator module 260 and the console communicator module 258 can allow for the catheter 202 to no longer be directly electrically connected to the system console 223 for data and/or power. For example, the catheter communicator module 260 can be wirelessly connected to the console communicator module 258 via a wireless connection. Examples of such a wireless connection can include a local area network (LAN), wide area network (WAN), personal area network (PAN), a distributed computing environment (e.g., a cloud computing environment), storage area network (SAN), Metropolitan area network (MAN), a cellular communications network, Long Term Evolution (LTE), visible light communication (VLC), Bluetooth, Worldwide Interoperability for Microwave Access (WiMAX), Near Field Communication (NFC), infrared (IR) communication, Public Switched Telephone Network (PSTN), radio waves, and/or the Internet, among other types of network relationships. Utilizing the wireless connection, the system console 223 can exchange data and/or power with the catheter 202 via the wireless connection.

In some examples, the catheter communicator module 260 exchanges only data with the console communicator module 258 of the system console 223 via the wireless connection. Data that is exchanged between the catheter 202 and the system console 223 can include device status information (e.g., catheter status information and/or system console status information), procedural data, device control signals, etc. For example, data that is exchanged between the catheter 202 and the system console 223 can include a catheter type of the catheter 202, a firing signal request, sensed pressures, among other types of data. The data can be transmitted from the catheter 202 to the system console 223 and/or from the system console 223 to the catheter 202.

The data can be exchanged between the system console 223 and the catheter 202 via a wireless data interface. Examples of the wireless data interface between the catheter communicator module 260 and the console communicator module 258 can include near field communication (NFC), Bluetooth, Bluetooth Low Energy (BLE), Wi-Fi, Zigbee, and/or adaptive network topology (ANT), among any other appropriate wireless data interfaces for wireless transmission of data.

In some examples, data can be exchanged between the catheter 202 and the system console 223 via at least two wireless data interfaces. Utilizing two different wireless data interfaces can improve the security of data transmission between the catheter 202 and the system console 223 as compared with utilizing a single wireless data interface. For example, data can be exchanged between the catheter communicator module 260 of the catheter 202 and the console communicator module 258 of the system console 223 using NFC and Bluetooth, among other example combinations.

In some examples, the catheter communicator module 260 exchanges only power with the console communicator module 258 of the system console 223 via the wireless connection. Power can be transmitted from the catheter 202 to the system console 223 and/or from the system console 223 to the catheter 202. For example, the system console 223 can wirelessly transmit power (e.g., 50 milliwatts (mW)) to the catheter 202 in order to power the light emitting diodes (LEDs) 269 included in the catheter 202 (e.g., included in the handle assembly 228 of the catheter 202) in order to indicate when the laser is activated.

Although the system console 223 is described as wirelessly transmitting 50 mW to the catheter 202, examples are not so limited. For example, the system console 223 can wirelessly transmit less than 50 mW to the catheter 202, or more than 50 mW to the catheter 202, for powering the LEDs 269 or for powering other components of the catheter 202.

Power can be exchanged between the system console 223 and the catheter 202 via a wireless power transfer technique. Examples of wireless power transfer techniques can include inductive coupling, such as Qi induction, WPC, NPC, etc.

In some examples, the catheter communicator module 260 exchanges both data and power with the console communicator module 258 of the system console 223 via the wireless connection. For example, the system console 223 can exchange power with the catheter 202 in order to power the LEDs 269 and the catheter 202 can exchange data with the system console 223 (e.g., to indicate sensed pressures during the medical procedure), among other examples.

As illustrated in FIG. 2, in some examples, the catheter 202 can include a catheter battery 268. In the embodiment illustrated in FIG. 2, the catheter battery 268 is illustrated as being located in the handle assembly 228. However, the catheter battery 268 can be located in any other location of the catheter 202.

In some examples, the catheter battery 268 can provide power to the catheter 202. For example, the catheter battery 268 can provide power to the LEDs 269 and/or other components of the catheter 202 in combination with or separate from the power provided wirelessly to the catheter 202 from the power source 225 of the system console 223. In some examples, the catheter battery 268 is rechargeable. For example, the catheter battery 268 may be recharged using power wirelessly received from the power source 225 of the system console 223. In some examples, the catheter battery 268 can be utilized for other purposes, such as providing power redundancy for the catheter system 200.

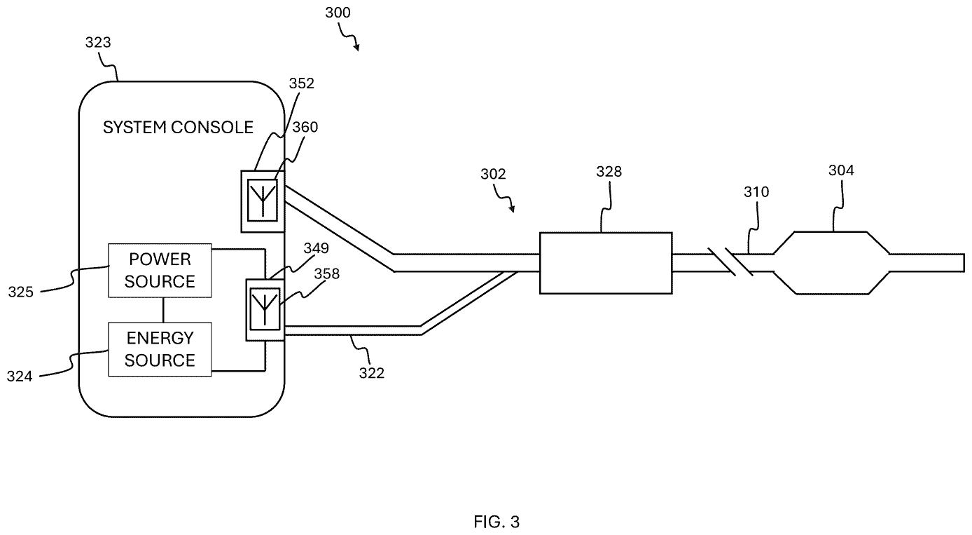

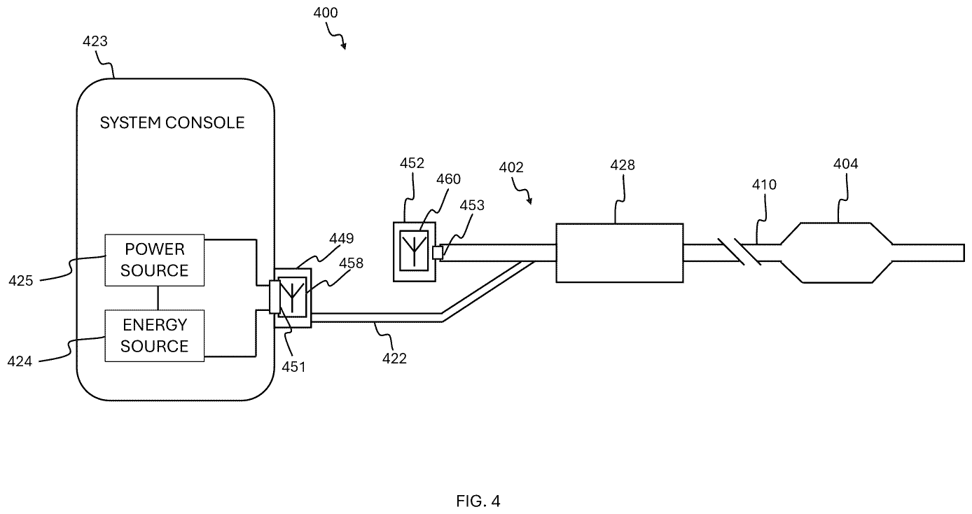

FIG. 3 is a simplified schematic view illustration of another embodiment of a portion of the catheter system including a catheter and a system console. The catheter 302 and the system console 323 can be the same and/or similar to the catheter 102 and the system console 123, previously described in connection with FIG. 1.

Similar to the system described in connection with FIG. 2, the system console 323 can include an energy source 324 and a power source 325. As previously mentioned in FIG. 1, the energy source 324 can be, for example, a laser. Additionally, the power source 325 can be electrically coupled to the energy source 324 and/or to a console connector 349. The console connector 349 can provide power to, for example, a console communicator module 358.

Similarly, the catheter 302 can include a shaft 310, a balloon 304 coupled with the shaft 310, and an energy guide (e.g., not illustrated in FIG. 3). The catheter 302 can be optically connected to the system console 323 via the energy guide bundle 322. The energy guide can receive energy from the energy source 324 of the system console 323 (e.g., via the energy guide bundle 322) and transmit the energy to an interior of the balloon 304 for treating the treatment site. For example, the catheter 302 can include an emitter (e.g., not illustrated in FIG. 3) coupled to the shaft 310 and to the energy guide. The emitter can transmit the energy from the energy source 324 into the interior of the balloon for treating the treatment site, as previously described in connection with FIG. 1.

In the embodiment illustrated in FIG. 3, the catheter communicator module 360 can be included in the system console 323. During use for a medical procedure, the energy guide bundle 322 of the catheter 302 can be connected (e.g., optically) to the system console 323 to the console connector 349. Additionally, the catheter 302 can be connected to the catheter connector 352 having the catheter communicator module 360, where the catheter connector 352 and the catheter communicator module 360 are included in the system console 323.

The catheter communicator module 360 can be wirelessly connected to the console communicator module 358. For example, the catheter 302 interfaces with a console interface (e.g., the catheter connector 352) that is connected to the catheter communicator module 360 in the system console 323. Therefore, while a wired connection between the catheter 302 and the system console 323 exists, the catheter 302 is wirelessly connected to the system console 323 via the console communicator module 358 (e.g., there is no direct wired electrical connection between the power source 325 and the catheter 302).

Utilizing the wireless connection, the system console 323 can exchange data and/or power with the catheter 302 via the wireless connection. In some examples, the catheter communicator module 360 exchanges only data with the console communicator module 358 of the system console 323 via the wireless connection. In some examples, the catheter communicator module 360 exchanges only power with the console communicator module 358 of the system console 323 via the wireless connection.

Data that is exchanged between the catheter 302 and the system console 323 can include device status information (e.g., catheter status information and/or system console status information), procedural data, device control signals, etc., similar to that described in connection with FIG. 2. Such data can be exchanged via a wireless data interface, or in some examples, via at least two wireless data interfaces.

Additionally, power can be exchanged between the system console 323 and the catheter 302 via a wireless power transfer technique. Examples of wireless power transfer techniques can include inductive coupling, such as Qi induction, WPC, NPC, etc., similar to that described in connection with FIG. 2.

As many catheters are single use, utilizing the embodiment described in FIG. 3 can allow for the wireless connection for data and/or power exchange between the system console 323 and the catheter 302 using an economically and environmentally friendly approach. For example, by placing the catheter communicator module 360 in the system console 323, physical waste is reduced (e.g., as opposed to the catheter communicator module 360 being located in the catheter 302, where the catheter communicator module

FIG. 4 is a simplified schematic view illustration of yet another embodiment of a portion of the catheter system including a catheter and a system console. The catheter 402 and the system console 423 can be the same and/or similar to the catheter 102 and the system console 123, previously described in connection with FIG. 1.

Similar to the system described in connection with FIG. 2, the system console 423 can include an energy source 424 and a power source 425. As previously mentioned in FIG. 1, the energy source 424 can be, for example, a laser. Additionally, the power source 425 can be electrically coupled to the energy source 424 and/or to a console connector 449. The console connector 449 can provide power to, for example, a console communicator module 458.

Similarly, the catheter 402 can include a shaft 410, a balloon 404 coupled with the shaft 410, and an energy guide (e.g., not illustrated in FIG. 4). The catheter 402 can be optically connected to the system console 423 via the energy guide bundle 422. The energy guide can receive energy from the energy source 424 of the system console 423 (e.g., via the energy guide bundle 422) and transmit the energy to an interior of the balloon 404 for treating the treatment site. For example, the catheter 402 can include an emitter (e.g., not illustrated in FIG. 4) coupled to the shaft 410 and to the energy guide. The emitter can transmit the energy from the energy source 424 into the interior of the balloon for treating the treatment site, as previously described in connection with FIG. 1.

In the embodiment illustrated in FIG. 4, the catheter 402 can include a catheter connector interface 453 at a proximal end of the catheter 402. The catheter connector interface 453 can be, for example, a data and/or power exchange interface. Such an interface can include Recommended Standard (RS) 232, RS485, controller area network (CAN) bus, Serial AT Attachment (SATA) bus, IEEE 1394 (e.g., FireWire), universal serial bus (USB) (and USB variants), pads and/or pogo pins that engage the pads, etc.

In the embodiment illustrated in FIG. 4, the catheter connector interface 453 can be connected to the catheter connector 452 including the catheter communicator module 460. In such an example, instead of the catheter connector interface 453 being connected to the console connector interface 451 of the system console 423, the catheter connector 452 can be connected to the catheter connector interface 453. That is, an existing catheter 402 can be retrofit with the catheter connector 452 having the catheter communicator module.

Additionally, the system console 423 can include a console connector interface 451. The console connector interface 451 can, for example, also be a data and/or power exchange interface, similar to the catheter connector interface 453 described above. In the embodiment illustrated in FIG. 4, the console connector interface 451 can be connected to the console connector 449 including the console communicator module 458. In such an example, instead of the console connector interface 451 being connected to the catheter connector interface 453, the console connector interface 451 can be connected to the console connector 449. That is, an existing system console 423 can be retrofit with the console connector 449 having the console communicator module 458. In this embodiment, the console connector 449 is still configured to be connected to the energy guide bundle 422 to directly optically connect the system console 423 with the catheter 402.

The catheter communicator module 460 can be wirelessly connected to the console communicator module 458. Therefore, the catheter 402 is wirelessly connected to the system console 423 via the console communicator module 458 (e.g., there is no direct wired electrical connection between the power source 425 and the catheter 402).

Utilizing the wireless connection, the system console 423 can exchange data and/or power with the catheter 402 via the wireless connection. In some examples, the catheter communicator module 460 exchanges only data with the console communicator module 458 of the system console 423 via the wireless connection. In some examples, the catheter communicator module 460 exchanges only power with the console communicator module 458 of the system console 423 via the wireless connection.

Data that is exchanged between the catheter 402 and the system console 423 can include device status information (e.g., catheter status information and/or system console status information), procedural data, device control signals, etc., similar to that described in connection with FIG. 2. Such data can be exchanged via a wireless data interface, or in some examples, via at least two wireless data interfaces.

Additionally, power can be exchanged between the system console 423 and the catheter 402 via a wireless power transfer technique. Examples of wireless power transfer techniques can include inductive coupling, such as Qi induction, WPC, NPC, etc., similar to that described in connection with FIG. 2.

As there are many catheter systems in use today, utilizing the embodiment described in FIG. 4 can allow for the retrofitting of a catheter communicator module 460 onto existing catheters 402 and a console communicator module 458 onto existing system consoles 423. The approach described in connection with FIG. 4 can then allow for the use of wireless exchange of data and/or power between existing catheters 402 and existing system consoles 423.

The present technology is also directed toward methods for treating a treatment site within or adjacent to a vessel wall, with such methods utilizing the devices disclosed herein.

In summary, based on the various embodiments of the present disclosure illustrated and described in detail herein, the catheter systems and related methods can include a catheter configured to advance to a vascular lesion, such as a calcified vascular lesion, or a fibrous vascular lesion, at a treatment site located within or adjacent a blood vessel within a body of a patient. The catheter includes a catheter shaft, and an inflatable balloon that is coupled and/or secured to the catheter shaft. The balloon can include a balloon wall that defines a balloon interior. The balloon can be configured to receive a catheter fluid within the balloon interior to expand from a deflated state suitable for advancing the catheter through a patient's vasculature, to an inflated state suitable for anchoring the catheter in position relative to the treatment site.

In certain embodiments, the catheter systems and related methods utilize an energy source, e.g., a light source such as a laser source or another suitable energy source, which provides energy that is guided by one or more energy guides, e.g., light guides such as optical fibers, which are disposed along the catheter shaft and within the balloon interior of the balloon to create a localized plasma in the catheter fluid that is retained within the balloon interior of the balloon. The energy guide can be used in conjunction with a plasma generator that is positioned at or near a guide distal end of the energy guide within the balloon interior of the balloon located at the treatment site. The creation of the localized plasma can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles (also sometimes referred to simply as “plasma bubbles”) can generate one or more pressure waves in the catheter fluid retained within the balloon interior of the balloon and thereby impart pressure waves onto and induce fractures in the vascular lesions at the treatment site within or adjacent to the blood vessel wall within the body of the patient. In some embodiments, the energy source can be configured to provide sub-millisecond pulses of energy, e.g., light energy, to initiate the plasma formation in the catheter fluid within the balloon to cause the rapid bubble formation and to impart the pressure waves upon the balloon wall at the treatment site. Thus, the pressure waves can transfer mechanical energy through an incompressible catheter fluid to the treatment site to impart a fracture force on the intravascular lesion. Without wishing to be bound by any particular theory, it is believed that the rapid change in catheter fluid momentum upon the balloon wall that is in contact with the intravascular lesion is transferred to the intravascular lesion to induce fractures to the lesion.

It should be noted that, as used in this specification and the appended claims, the singular forms “a,” “an,” and “the” include plural referents unless the content and/or context clearly dictates otherwise. It should also be noted that the term “or” is generally employed in its sense, including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

It is recognized that the figures shown and described are not necessarily drawn to scale, and that they are provided for ease of reference and understanding, and for relative positioning of the structures.

The headings used herein are provided for consistency with suggestions under 37 CFR 1.77 or otherwise to provide organizational cues. These headings shall not be viewed to limit or characterize the disclosure(s) set out in any claims that may issue from this disclosure. As an example, a description of a technology in the “Background” is not an admission that technology is prior art to any disclosure(s) herein.

The embodiments described herein are not intended to be exhaustive or to limit the disclosure to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present disclosure.

While a number of illustrative aspects and embodiments of the catheter systems have been discussed above, those of skill in the art will recognize certain modifications, permutations, additions, and sub-combinations thereof. It is, therefore, intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. A catheter for treating a treatment site within or adjacent to a vessel wall of a blood vessel or a heart valve within a body of a patient, the catheter comprising:
a shaft;
a balloon coupled with the shaft;
an energy guide extending through the shaft and into the balloon that receives energy from an energy source;
an emitter coupled to the shaft and the energy guide, wherein the emitter is configured to transmit the energy from the energy source into an interior of the balloon for treating the treatment site; and
a catheter communicator module configured to be wirelessly connected to a system console via a wireless connection such that the catheter exchanges at least one of data and power with the system console via the wireless connection.

2. The catheter of claim 1, wherein the catheter communicator module is configured to exchange only data with the system console via the wireless connection.

3. The catheter of claim 1, wherein the catheter communicator module is configured to exchange only power with the system console via the wireless connection.

4. The catheter of claim 1, wherein the catheter communicator module is configured to exchange data and power with the system console via the wireless connection.

5. The catheter of claim 1, wherein the catheter further includes a catheter battery.

6. The catheter of claim 5, wherein the catheter battery is configured to provide power to the catheter.

7. The catheter of claim 5, wherein the catheter battery is rechargeable.

8. A system console for treating a treatment site within or adjacent to a vessel wall of a blood vessel or a heart valve within a body of a patient, the system console comprising:
a power source configured to provide power;
an energy source configured to receive energy from the power source, wherein the energy source is configured to transmit energy to an energy guide of a catheter for treating the treatment site; and
a console communicator module configured to be wirelessly connected to a catheter communicator module of the catheter via a wireless connection such that the system console is configured to wirelessly exchange at least one of data and power with the catheter via the wireless connection.

9. The system console of claim 8, wherein the console communicator module is configured to exchange only data with the catheter via the wireless connection.

10. The system console of claim 8, wherein the console communicator module is configured to exchange only power with the catheter via the wireless connection.

11. The system console of claim 8, wherein the console communicator module is configured to exchange data and power with the catheter via the wireless connection.

12. A catheter system for treating a treatment site within or adjacent to a vessel wall of a blood vessel or a heart valve within a body of a patient, the catheter system comprising:
a system console including an energy source and a console communicator module; and
a catheter that includes a shaft, a balloon coupled with the shaft, and an energy guide, wherein:
the energy guide receives energy from the energy source and transmits the energy to an interior of the balloon for treating the treatment site; and
a catheter communicator module is wirelessly connected to the console communicator module of the system console via a wireless connection such that the system console exchanges at least one of data and power with the catheter via the wireless connection.

13. The catheter system of claim 12, wherein the catheter communicator module is included in the catheter.

14. The catheter system of claim 12, wherein:
the console communicator module is a tuned coil; and
the catheter communicator module is an antenna.

15. The catheter system of claim 12, wherein:
the catheter communicator module is included in the system console; and
the catheter is configured to interface with a console interface connected to the catheter communicator module in the system console.

16. The catheter system of claim 12, wherein the data is exchanged via a wireless data interface including at least one of:
near field communication (NFC);
Bluetooth;
Bluetooth Low Energy (BLE);
Wi-Fi;
Zigbee; and
Adaptive Network Topology (ANT).

17. The catheter system of claim 12, wherein the data is exchanged via at least two wireless data interfaces.

18. The catheter system of claim 12, wherein the power is exchanged via a wireless power transfer technique.

19. The catheter system of claim 12, wherein:
the catheter includes a catheter connector interface at a proximal end of the catheter; and
the system console includes a console connector interface.

20. The catheter system of claim 19, wherein:
the catheter communicator module is connected to the catheter connector interface; and
the console communicator module is connected to the console connector interface.
